(19) United States
(12) Patent Application Publication
(10) Pub. No.: US 2022/0175648 A1
(43) Pub. Date: Jun. 9, 2022

(54) TOPICAL FORMULATIONS WITH LIPID-BASED CARRIERS

(71) Applicant: KATE ROSE LLC, Bethesda, MD (US)

(72) Inventors: Katie Elizabeth ROSENBERG, Bethesda, MD (US); Thomas Michael HERRERA, Bethesda, MD (US)

(73) Assignee: KATE ROSE LLC, Bethesda, MD (US)

(21) Appl. No.: 17/116,776

(22) Filed: Dec. 9, 2020

(51) Int. Cl. A61K 8/67; A61K 9/127; A61K 8/9783; A61Q 19/00

(52) U.S. Cl. CPC A61K 8/676; A61K 9/1274; A61Q 19/00; A61K 8/9783; A61K 8/678

(57) ABSTRACT

In some embodiments, a formulation may include one or more of a lipid-soluble vitamin C compound, derivative, or analog, a cinnamic acid derivative, one or more lipid-soluble vitamin E compounds, and a lipid-based carrier. In some embodiments, the cinnamic acid may include one or more of p-coumaric acid, ferulic acid, caffeic acid, sinapinic acid, combinations thereof, and cis and trans isomers thereof. In some embodiments, the vitamin E compounds may alpha, beta, delta and gamma tocotrienols.

### FIELD OF THE INVENTION

The present invention relates to the field of cosmetics and dermatological compositions for the improvement of photo-aging skin.

### BACKGROUND

Cosmetics are a multi-billion dollar per year industry in the United States alone. Skincare is one of the fastest growing markets within this vast industry. Current trends show that the anti-aging segment has further increased industry revenue. Antioxidants such as, vitamin C and vitamin E, are cornerstones in many of these anti-aging products.

Antioxidants are important in skincare due to their ability to fight free radicals. Free radicals are generated from UV radiation, pollutants, alcohol, and other toxins. Free radicals are unstable molecules that are highly reactive and lead to oxidative damage of cells. Oxidative stress plays an integral role in premature aging, hyperpigmentation, loss of skin elasticity, as well as leaving the skin more prone to acne and eczema. Antioxidants are able to donate their electrons to free radicals, which neutralizes the negative effects. Antioxidants may vary by their potency, solubility, as well as which reactive species they are able to scavenge. For example, vitamin E is known to scavenge peroxyl radicals, which helps protect against lipid peroxidation of cell membranes. However, more lipid soluble vitamin E isomers, such as delta- and gamma-tocotrienol, have shown to have increased potency and improved ability to scavenge peroxyl radicals.

Vitamin E is an essential, fat soluble vitamin that is found in many vegetables, nuts, and oils. Vitamin E is an antioxidant that is used to nourish and protect the skin from free radicals and sun damage which are associated with signs of aging, skin cancer, skin irritation or inflammation, dermatitis, allergy, psoriasis, acne, eczema, rosacea, and other dermatologic skin conditions. It is also said to improve wound healing and scar formation. However, not all vitamin E is equal.

Vitamin E is not a single compound. It is composed of eight isomers classified under the term tocochromanols: alpha-, beta-, delta-, and gamma-tocopherol (T1), and alpha-, beta-, delta-, and gamma-tocotrienol (as used herein, the term “T3” refers to any or all of these tocotrienol isomers). The key structural differences between the two groups are their molecular head and isoprenoid tail structures. Tocopherols have a saturated isoprenoid tail, whereas tocotrienols have an unsaturated isoprenoid tail and a more lipophilic head. The tail structure affects the antioxidant activity of the isomer and the head structure affects the penetration into cellular membranes. Accordingly, there is a need for a more effective topical vitamin E with having a molecular structure that will provide improved antioxidant activity and penetration when used by itself or when formulated in products, including but not limited to cosmetics and pharmaceuticals.

Vitamin C is an antioxidant that has photo-protective effects against UV A and UV B, fights free radicals that lead to oxidative damage to the skin, and increases collagen production to improve signs of aging such as fine lines and wrinkles. The most commonly used form of vitamin C in cosmetic formulations is L-ascorbic acid. There are several problems with the topical delivery of L-ascorbic acid. It is a water soluble molecule that is unable to penetrate the epidermis at the skin's natural pH (pH 4-5). It requires a more acidic pH of <3.5 in order to penetrate the skin, which may lead to skin irritation and leave the skin more acne prone. Additionally, L-ascorbic acid oxidizes and degrades easily. Degradation is increased when it is exposed to high temperatures, high or neutral pH, or exposed to light and air leading to a short shelf-life. L-ascorbic acid must be in an aqueous solution, which requires added preservatives in order to prevent the growth of bacteria and fungus in the solution. Consequently, there is a need for a topical vitamin C and vitamin E formulation that is preservative-free, has increased stability, has increased penetration to the dermis, and works at the skin's natural pH to reduce the risk of adverse skin reactions.

Cinnamic acid derivatives, such as p-coumaric acid, ferulic acid, caffeic acid, sinapinic acid, have been shown to protect the skin and have significant benefits. Ferulic acid is an antioxidant that is naturally found in the cell wall of plants including bran and certain fruit seeds. When applied topically, ferulic acid helps protect the skin from free radicals and oxidative stress. However, when applied by itself, it only provides partial protection. It works synergistically with other antioxidants such as vitamin C and E to further protect the skin against harmful effects of free radicals. Ferulic acid is insoluble in aqueous solutions. Ferulic acid requires a solvent, such as methanol, ethanol, isopropanol, ethylene glycol, ethylene acetate, dimethyl sulfoxide (DMSO), and propylene glycol, in order to dissolve in an aqueous solution. These chemicals are harsh on the skin and promote dehydration, which may lead to more visible pores and wrinkles. There is a need for a ferulic acid composition that does not require an alcohol solvent.

Current vitamin C, vitamin E, and ferulic acid compositions are aqueous solutions that use water as the carrier. As previously described, water is a poor carrier due to the inability to deliver its solvents through the epidermis without added chemicals and a more acid pH. Consequently, there is a need for a lipid soluble carrier that can effectively penetrate the epidermis at the skin's natural pH (pH 4-5) without added chemicals. Furthermore, there is a need for a lipid soluble carrier that can be used by itself or formulated into other products, including but not limited to cosmetics and pharmaceuticals, which has optimal absorption and allows for maximal penetration of vitamin C and E into the dermis while being tolerable for all skin types.

### SUMMARY

The following presents a simplified summary in order to provide a basic understanding of some aspects described herein. This summary is not an extensive overview of the claimed subject matter. It is intended to neither identify key or critical elements of the claimed subject matter nor delineate the scope thereof. Its sole purpose is to present some concepts in a simplified form as a prelude to the more detailed description that is presented later.

The present invention addresses solutions to the said problems by creating a formulation that may provide increased antioxidant activity and efficacy, increased penetration of vitamins C, E, and ferulic acid into the skin, and increased stability and shelf-life. In some embodiments, this may be accomplished with the use of only four ingredients. In some embodiments, added preservatives and alcohol solvents may be avoided, thus providing additional benefits relative to conventional vitamin C formulations. In some embodiments, the present invention may work at the skin's natural pH to penetrate human skin, significantly decreasing the likelihood of adverse skin reactions, such as skin irritation, acne, wrinkles, and dehydration.

In some embodiments, a formulation may include one or more of a lipid-soluble vitamin C compound, derivative, or analog, a cinnamic acid derivative, one or more lipid-soluble vitamin E compounds, and a lipid-based carrier. In some embodiments, the cinnamic acid may include one or more of p-coumaric acid, ferulic acid, caffeic acid, sinapinic acid, combinations thereof, and cis and trans isomers thereof. In some embodiments, the vitamin E compounds may alpha, beta, delta and gamma tocotrienols.

Further variations are described in the detailed description of the invention below.

### DETAILED DESCRIPTION

While aspects of the subject matter of the present disclosure may be embodied in a variety of forms, the following description and accompanying drawings are merely intended to disclose some of these forms as specific examples of the subject matter. Accordingly, the subject matter of this disclosure is not intended to be limited to the forms or embodiments so described and illustrated.

Structural differences between tocopherols and tocotrienols influence their respective efficacy when used as antioxidants. Alpha-tocopherol has a relatively high level of bioavailability and is present in many common vegetable oils. Tocotrienols are less common and are only found in select sources. The largest content of tocotrienols are found in rice bran oil (25% T3: 75% T1), palm oil (50% T3: 50% T1), and annatto seeds (99.9% T3: 0.01% T1). Annatto is a red-orange seed derived from the achiote tree. The color of annatto comes from various carotenoid pigments, mainly bixin and norbixin, found in the waxy coating of the seeds. The pigment is isolated and often used to impart a yellow or orange color to foods, fabrics, and cosmetics. Until recently, the lipids from the annatto seed (including the vitamin E) have been discarded as waste.

Annatto contains rare vitamin E isomers—gamma- and delta-tocotrienol—that have greater antioxidant activity as compared to alpha-tocopherol, the most widely used and commercially available vitamin E isomer. Specifically, annatto contains 100% tocotrienols (90% delta and 10% gamma isomers), with virtually no tocopherols.

Due to the rarity of T3 isomers, the benefits of T3 have been widely overlooked. Research of tocotrienols only makes up about 1% of all vitamin E literature, and this research has been limited to dietary implications of annatto, rather than the potential for annatto to be used in topical applications. Consequently, the benefits of topical annatto oil have not been previously studied.

Delta- and gamma-tocotrienol, have 40-60 times more antioxidant activity than alpha-tocopherol, possibly due to the unsaturated tail structure of T3. In addition, T3 has increased penetration into the cell membrane of skin cells, which may be due to the more lipophilic head structure. Alpha-tocopherol has higher affinity than tocotrienols, which leads to competitive inhibition of tocotrienols when placed in a mixture. Optimal tocotrienol preparations for use in, e.g., skin-care and haircare products, should contain less than 35% tocopherol (preferably less than 25%, and more preferably less than 15%, 5%, or 1% tocopherol) and more than 40% tocotrienol (preferably more than 50%, and more preferably more than 60%, 70%, 80%, or 90% tocotrienol) to avoid the competitive inhibition. In some embodiments, the tocotrienol component may include one or both of gamma- and delta-tocotrienol.

The most commonly used form of vitamin C in cosmetic formulations is L-ascorbic acid. It is the natural form of vitamin C. L-ascorbic acid is water soluble and is often formulated in aqueous solutions. Due to its water soluble properties, it is unable to penetrate the lipophilic epidermis. In order for L-ascorbic acid to penetrate the epidermis, the pH of the solution must be <3.5, which is more acidic than the pH of normal human skin (pH 4-5). The more acidic pH impairs the barrier function of the epidermis which allows for only a limited amount of the L-ascorbic acid to reach the dermis (deeper layer of the skin). When the pH of skin is more acidic than its normal state, the skin may be susceptible to inflammatory skin conditions, including eczema and acne. Therefore, applying vitamin C with a pH of <3.5 may lead to skin irritation and acne. Additionally, studies show that L-ascorbic acid is unstable and susceptible to degradation when exposed to light or air. Its instability leads to a short shelf-life. Studies show that L-ascorbic acid breaks down by 20% after just one year. Aqueous solutions of L-ascorbic acid also require added preservatives in order to prevent growth of bacteria and fungus.

There are many derivatives and analogs of L-ascorbic acid. These analogs differ in their chemical properties which alters the solubility and stability of the molecule. Tetrahexyldecyl ascorbate (THD) is an analog of vitamin C that has four isopalmitic acid esters located at the 2, 3, 5, and 6 carbon positions. It has been shown to convert to L-ascorbic acid in vivo, which leads to the same physiologic benefits of L-ascorbic acid. Additionally, it has shown to efficiently penetrate the dermis and convert to L-ascorbic acid without added chemicals. THD is most effective at pH <5, which is comparable to normal human skin (pH 4-5). In vitro studies show that THD is effective at protecting against UV B and reactive oxygen species. THD has shown to increase collagen synthesis up to two times greater than L-ascorbic acid at the same dosage.

Topical L-ascorbic acid combined with alpha-tocopherol (vitamin E) has been shown to have a synergistic effect against UV-induced erythema and oxidative damage. L-ascorbic acid is able to recycle photo-oxidized vitamin E which may account for the synergistic effect when combined in solution. As previously described, THD is converted to L-ascorbic acid which combined with gamma- and delta-tocotrienol may further potentiate the recycling of oxidized molecules leading to a further synergistic effect against UV and oxidative damage. Additionally, studies show that a 10% solution of THD decreases the presence of age spots. This is likely due to the increased penetration and efficient conversion to L-ascorbic acid at the dermal layer. Overall, THD is a lipid-soluble vitamin C analog that has shown to have equivalent or even improved physiologic effects of L-ascorbic acid, increased penetration, and increased stability without the need for added chemicals.

Ferulic acid is an antioxidant that works synergistically with other antioxidants such as vitamin C and E, which further protects the skin against harmful effects of free radicals. Ferulic acid is insoluble in aqueous solutions. Ferulic acid requires a solvent, such as methanol, ethanol, isopropanol, ethylene glycol, ethylene acetate, dimethyl sulfoxide (DMSO), and propylene glycol, in order to dissolve in an aqueous solution. These chemicals are harsh on the skin and promote dehydration, which may lead to more visible pores and wrinkles. Ferulic acid is weakly soluble in lipid-based solutions. In some embodiments, ferulic acid may be soluble in an amount by weight of approximately 0.2% in a lipid-based solution. In some embodiments, this solubility may be achieved without added chemicals. Therefore, ferulic acid may be placed in a lipid-based vitamin C and vitamin E solution without the need for added solvents.

Jojoba (Simmondsia chinesis) seed oil is a plant wax that comes from the jojoba plant. The terms “jojoba oil” and “jojoba wax” are often used interchangeably because the wax is liquid at room temperature. It is composed almost entirely of mono-esters of long-chain fatty acids and alcohols (wax ester), accompanied by only a tiny fraction of triglyceride esters. This composition accounts for its extreme shelf-life stability and extraordinary resistance to high temperatures, compared with true vegetable oils. Its great stability and rancid resistant properties make it a dependable, natural preservative. In addition, jojoba oil is an emollient that closely resembles human sebum, the skin's natural oils. It has shown to decrease natural skin oils on oily skin and to moisturize dry skin. Therefore, it is suitable for all skin types. It is also considered non-comedogenic, meaning it will not clog hair follicles and lead to acne.

Moreover, jojoba oil is a carrier oil, also known as a base oil. It has little to no scent and is commonly used to dilute essential oils and absolutes before they are applied to the skin. It is so named a carrier oil because it is said to “carry” the essential oil (or other oils) through the water-resistant epidermis and into the dermal skin layer. When jojoba oil is combined with a lipid-soluble vitamin E and vitamin C solution, it may more efficiently carry the antioxidants to the dermal layer. In some embodiments, jojoba oil may deliver antioxidants to the dermal layer at the skin's natural pH and without added chemicals. Additionally, jojoba oil is a natural preservative and does not require added preservatives.

Because vitamin C, vitamin E, and ferulic acid operate synergistically, it would be desirable to combine these components in a formulation that can be used, e.g., topically on a user's skin. Prior to the instant invention, however, no such formulation has been achieved that effectively promotes the penetration of the active components into a user's skin and does so at a pH that is comparable to the natural pH of human skin. This is, in part, because the natural form of vitamin C (L-ascorbic acid) is water-soluble and unable to penetrate the lipophilic epidermis unless it is provided in a solution having a pH less than 3.5. Further, even when formulated as such, the penetration of vitamin C and vitamin E is suboptimal, and the relatively acidic formulation can damage the user's skin.

These shortcomings may be overcome by using a lipid-based formulation that more readily penetrates the epidermis. Because the natural form of vitamin C is water-soluble, a lipid-soluble analog of vitamin C may instead be selected for the formulation. For example, THD is a lipid-soluble analog of vitamin C that has been shown to convert to L-ascorbic acid in vivo, which leads to the same physiologic benefits of L-ascorbic acid. THD also efficiently penetrates the dermis and converts to L-ascorbic acid at a pH comparable to that of human skin and without added chemicals. In some embodiments, a lipid-soluble analog may be dissolved in a lipid-based carrier. In some embodiments, the lipid-based carrier may include one or more liquid wax esters or derivatives, such as jojoba oil. In other embodiments, the lipid-based carrier may include one or more triglyceride esters or derivatives. The one or more triglyceride esters may include rice bran oil, palm oil, tea tree oil, grapeseed oil, argan oil, black currant oil, rosehip seed oil, chamomile oil or other triglyceride esters or derivatives

The formulation may be further improved by providing vitamin E compounds with relatively high solubility in lipid-based carriers and improved penetration in the epidermis. For example, T3 vitamin E isomers have lipophilic head structures which improve penetration into the cell membrane of skin cells. To reduce or minimize competitive inhibition between tocopherols and tocotrienols, a formulation may contain less than 35% tocopherol (preferably less than 25%, and more preferably less than 15%, 5%, or 1% tocopherol) and more than 40% tocotrienol (preferably more than 50%, and more preferably more than 60%, 70%, 80%, or 90% tocotrienol).

A lipid-based formulation has further advantages in that ferulic acid is weakly soluble in lipid-based solutions whereas it is insoluble in aqueous solutions. When used in aqueous solutions, ferulic acid requires a solvent, such as methanol, ethanol, isopropanol, ethylene glycol, ethylene acetate, dimethyl sulfoxide (DMSO), and propylene glycol, which can be harsh on the skin and promote dehydration. By instead providing a lipid-based formulation containing ferulic acid, these solvents may be beneficially avoided. For example, a lipid-based formulation may include only, or consist essentially of, a lipid-soluble vitamin C analog, vitamin E isomers (preferably, relatively lipid-soluble isomers), and a cinnamic acid derivative, each dissolved in a lipid-based carrier without any added chemicals, solvents, or preservatives. By using a lipid-based formulation as disclosed herein, it is therefore possible to maximize the beneficial effects of the formulation and while eliminating chemicals having adverse effects.

In some embodiments, an exemplary formulation may be a single-phase anhydrous solution composition. The formulation may include tetrahexyldecyl ascorbate or other lipid soluble vitamin C derivatives and analogs. In some embodiments, tetrahexyldecyl ascorbate may comprise 1% to 20% of the formulation by weight. The formulation may also include one or more cinnamic acid derivatives, such as p-coumaric acid, ferulic acid, caffeic acid, sinapinic acid, combinations thereof, and cis and trans isomers thereof. The one or more cinnamic acid derivatives may comprise 0.1% to 5.0% of the formulation by weight. The formulation may further include one or more vitamin E isomers. For example, the formulation may include alpha, beta, delta and/or gamma tocotrienols, and combinations thereof. In some embodiments, the vitamin E isomers may comprise 0.2 to 4% of the formulation by weight. In some embodiments, tocotrienols may comprise 5% to 100% of the vitamin E isomers included in the formulation. In some embodiments, the formulation may further comprise a lipid-based carrier. For example, the lipid-based carrier may include one or more liquid wax esters or triglyceride esters. In some embodiments, the liquid-based carrier may be jojoba oil. In some embodiments, the lipid-based carrier may include one or more of rice bran oil, palm oil, tea tree oil, grapeseed oil, argan oil, black currant oil, rosehip seed oil, chamomile oil or other triglyceride esters or derivatives.

In some embodiments, a tetrahexyldecyl ascorbate (vitamin C), delta- and gamma-tocotrienol vitamin E, ferulic acid, and Simmondsia chinesis (jojoba) seed oil composition may be provided in a single-phase anhydrous solution. This solution may be created by dissolving the ferulic acid in jojoba oil. In some embodiments, the ferulic acid may be dissolved in the jojoba oil at a temperature between 10 degrees Celsius to 40 degrees Celsius. In some embodiments, the solution may be simultaneously mixing with a magnetic stirrer. Once the ferulic acid has dissolved into the jojoba oil, one or both of liquid tetrahexyldecyl ascorbate (vitamin C) and liquid vitamin E (delta- and gamma-tocotrienol) may be added to the solution. The solution may be mixed using a magnetic stirrer at room temperature until a homogenous solution is created.

The present invention relates to improved formulations that provide enhanced stability, enhanced solubility, increased antioxidant activity, and an enhanced photo-protective effect as compared to prior compositions. Additionally, the disclosed solutions do not require added preservatives, alcohol solvents, and works at the skin's natural pH.

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims.

1. A formulation comprising:
a lipid-soluble vitamin C compound, derivative, or analog thereof;
a cinnamic acid derivative selected from a group consisting of: p-coumaric acid, ferulic acid, caffeic acid, sinapinic acid, combinations thereof, and cis and trans isomers thereof;
one or more vitamin E compounds selected from a group consisting of: alpha, beta, delta and gamma tocotrienols; and
a lipid-based carrier.

2. The formulation of claim 1, wherein the formulation is a single-phase anhydrous solution.

3. The formulation of claim 1, wherein the lipid-based carrier comprises a liquid wax ester or derivative thereof.

4. The formulation of claim 3, wherein the liquid wax ester comprises jojoba oil.

5. The formulation of claim 1, wherein the lipid-based carrier comprises a triglyceride ester or derivative thereof selected from a group consisting of: rice bran oil, palm oil, tea tree oil, grapeseed oil, argan oil, black currant oil, rosehip seed oil, chamomile oil or other triglyceride esters or derivatives.
